(19) United States
(12) Patent Application Publication
(10) Pub. No.: US 2024/0285885 A1
(43) Pub. Date: Aug. 29, 2024

(54) OXYGEN DELIVERY AND RECLAMATION SYSTEM

(71) Applicants: Paragon Space Development Corporation, Tucson, AZ (US); Giner, Inc., Newton, MA (US)

(72) Inventors: Patrick Dee Pasadilla, Tucson, AZ (US); Peter Francis Readey, Lebanon, TN (US); Robert Jacobi, Tucson, AZ (US); Samuel Zachary Lynn, Somerville, MA (US); Judith Lattimer, Cambridge, MA (US)

(21) Appl. No.: 18/574,203

(22) PCT Filed: Jun. 30, 2022
(86) PCT No.: PCT/US2022/035616

§ 371 Date: Dec. 26, 2023

Related U.S. Application Data
(60) Provisional application No. 63/202,987, filed on Jul. 2, 2021.

(51) Int. Cl. A61M 16/00; A61M 16/06; A61M 16/10; A61M 16/20; B63C 11/24

(52) U.S. Cl. CPC A61M 16/0045; A61M 16/0009; A61M 16/0057; A61M 16/0078; A61M 16/0093; A61M 16/06; A61M 16/101; A61M 16/202; B63C 11/24; A61M 2016/1025

(57) ABSTRACT

This disclosure provides an oxygen delivery and reclamation system that supplies breathable oxygen-enriched gas to one or more users in a chamber. The one or more users receive oxygen at specified oxygen level setpoints. Exhalation air from the one or more users may be purged from the breathing loop using one or both of a recycle pump and an electronically actuated valve. Exhalation air in a purge line and optionally chamber air pulled from the chamber may be recycled using an oxygen concentrator. The oxygen concentrator generates oxygen-enriched gas and oxygen-depleted gas, where the oxygen-enriched gas may be delivered back to one or more users in the chamber.

### INCORPORATION BY REFERENCE

A PCT Request Form is filed concurrently with this specification as part of the present application. Each application that the present application claims benefit of or priority to as identified in the concurrently filed PCT Request Form is incorporated by reference herein in its entirety and for all purposes.

### STATEMENT REGARDING FEDERALLY SPONSORED RESEARCH OR DEVELOPMENT

Some embodiments of this invention were made with United States Government Support under Contract No. N6833520C0187 awarded by the Naval Sea Systems Command. The U.S. Government has certain rights in this invention.

### TECHNICAL FIELD

This disclosure relates to oxygen supply systems, devices, apparatuses, and methods, and more particularly to systems for reclamation and delivery of breathable oxygen-enriched gas to divers, miners, patients, pilots, or other users in an enclosed space.

### BACKGROUND

Oxygen delivery is crucial in a number of life support systems. In medicine, oxygen therapy is used to treat ailments such as emphysema, pneumonia, and some heart disorders. Oxygen can be delivered in a number of ways including a nasal cannula, face mask, and a hyperbaric treatment chamber. Hyperbaric treatment chambers are specialized chambers that increase the partial pressure of oxygen around the patient and can treat conditions such as carbon monoxide poisoning and decompression sickness (i.e., the “bends”). With Covid-19 and symptoms of acute respiratory distress syndrome, receiving supplemental oxygen delivery can be life-saving for patients.

Scuba divers and submariners often rely on artificially delivered oxygen. Underwater diving or rebreather systems typically utilize an oxygen supply system for delivery of oxygen or oxygen-enriched gas to a diver, where the flow of oxygen or oxygen-enriched gas can be adjusted. The partial pressure of oxygen (PPO₂) may be controlled as depths vary. When divers or submariners go to depths where they are exposed to elevated atmospheric pressure for long periods of time, they are prone to develop decompression sickness upon returning to normal atmospheric pressures. Miners emerging from a mine may also experience decompression sickness. With decompression sickness, bubbles of inert gas can occur in a person's body as a result of pressure reduction during ascent. To increase the rate of decompression, decompression chambers may supply oxygen or oxygen-enriched gas to divers or submariners to mitigate the risk of developing decompression sickness.

In addition to mining, diving, and submarining applications, oxygen may be very important to mountain climbers, aviators, and high-altitude parachutists. At reduced atmospheric pressures, air is less dense and less oxygen enters the lungs. The situation can result in a deficiency of oxygen in the blood, or hypoxemia. This can result in heavy breathing, lightheadedness, euphoria, overconfidence, apathy, fatigue, visual disturbances, chest pain, unconsciousness, seizures, and even death. Hypoxemia can be ameliorated by delivery of supplemental oxygen.

### SUMMARY

The systems, methods and devices of this disclosure each have several innovative aspects, no single one of which is solely responsible for the desirable attributes disclosed herein.

One innovative aspect of the subject matter described in this disclosure can be implemented in an oxygen delivery and reclamation system. The system can include one or more rebreather devices in a chamber, each of the one or more rebreather devices comprising a mask configured to be worn by a user. The system can further include an oxygen-enriched gas supply source configured to supply an oxygen-concentrated inhalation gas upon activation to the one or more rebreather devices, a purge line configured to purge exhalation gas from the one or more rebreather devices, a recycle line coupled to the purge line and configured to receive the exhalation gas from the purge line, and an oxygen concentrator coupled to the recycle line, where the oxygen concentrator is configured to receive the exhalation gas and output an oxygen-enriched gas to the oxygen-enriched gas supply source and an oxygen-depleted gas to an ambient environment outside the chamber, to a storage source, or to the chamber.

In some implementations, the oxygen-enriched gas comprises at least 90% oxygen by volume. In some implementations, the oxygen delivery and reclamation system further includes a carbon dioxide (CO₂) scrubber coupled to the recycle line, where the carbon dioxide scrubber is configured to remove carbon dioxide from the exhalation gas prior to being received by the oxygen concentrator. In some implementations, the oxygen delivery and reclamation system further includes a chamber pump configured to supply chamber air from the chamber to the recycle line, where the oxygen concentrator is configured to receive the chamber air or a mixture of the chamber air and the exhalation gas and output the oxygen-enriched gas and the oxygen-depleted gas. In some implementations, the oxygen delivery and reclamation system further includes a controller configured with instructions to perform the following operations: receive an indication that an ambient pressure or oxygen concentration/partial pressure of the chamber is above a threshold value, and actuate an electronically or mechanically actuated valve coupled to the chamber pump to pull the chamber air into the recycle line and regulate the ambient pressure. In some implementations, the oxygen delivery and reclamation system further includes a purge pump configured to generate a vacuum in the recycle line and pull the exhalation gas from the purge line to the recycle line. In some implementations, the oxygen delivery and reclamation system further includes an atmospheric line coupled to the purge line and configured to vent the exhalation gas to the ambient environment outside the chamber or to the storage source, where the exhalation gas is supplied to the recycle line or the atmospheric line via an electronically or mechanically actuated valve. In some implementations, the chamber is a pressurized vessel. In some implementations, the oxygen delivery and reclamation system further includes an oxygen tank coupled to the oxygen-enriched gas supply source and configured to supply oxygen to the oxygen-enriched gas supply source, where the oxygen-concentrated inhalation gas includes the oxygen or a mixture of the oxygen and the oxygen-enriched gas. In some implementations, each of the one or more rebreather devices further comprises: an inhale breathing bag coupled to an inlet of the mask, and an exhale breathing bag coupled to an outlet of the mask. In some implementations, the oxygen delivery and reclamation system further includes a controller configured with instructions to perform the following operations: receive an input for a desired oxygen concentration to be supplied to a user of one of the one or more rebreather devices, and actuate an oxygen intake valve fluidly coupled to the oxygen-enriched gas supply source and a chamber air intake valve fluidly coupled to the chamber to deliver the desired oxygen concentration to a rebreather device associated with a user. In some implementations, the controller is further configured with instructions to perform the following operation: actuate a purge valve coupled to the purge line, independent of user lung capacity and lung strength, to purge the exhalation gas independently from the one or more rebreather devices. In some implementations, the one or more rebreather devices comprises a plurality of rebreather devices comprising masks worn by a plurality of users. In some implementations, the oxygen concentrator is an electrochemical oxygen concentrator. In some implementations, the oxygen delivery and reclamation system further includes a hygiene/trace contaminant removal device coupled to the purge line and configured to remove contaminants from the exhalation gas prior to reaching the recycle line.

Another innovative aspect of the subject matter described in this disclosure can be implemented in a method of delivering and reclaiming oxygen. The method includes receiving an input for a desired oxygen concentration to be supplied to a user of a rebreather device inside a chamber, delivering an inhalation gas to the user at the desired oxygen concentration from an oxygen-enriched gas supply source, purging exhalation gas from the rebreather device by a vacuum generated in a purge line coupled to the rebreather device, recycling the exhalation gas to an oxygen concentrator, and producing, by the oxygen concentrator, an oxygen-enriched gas that is supplied to the oxygen-enriched gas supply source.

In some implementations, the method further includes producing, by the oxygen concentrator, an oxygen-depleted gas that is delivered to an ambient environment outside the chamber, to a storage source, or to the chamber. In some implementations, the method further includes pulling chamber air from the chamber into a recycle line fluidly coupled to the oxygen concentrator, and supplying the chamber air to the oxygen concentrator, wherein the oxygen-enriched gas is produced by one or both of the chamber air and the exhalation gas. In some implementations, the oxygen-enriched gas comprises at least 90% oxygen by volume. In some implementations, the method further includes venting the exhalation gas from the rebreather device to an ambient environment outside the chamber or to a storage source.

Another innovative aspect of the subject matter described in this disclosure can be implemented in a method of monitoring and controlling gas composition delivery. The method includes receiving, at a computer device, a desired target for a concentration/partial pressure of a first gas to be delivered to a user of a gas control system, and receiving, at the computing device, data regarding a concentration/partial pressure of the first gas in the gas control system, a concentration/partial pressure of a second gas in the gas control system, and one or more internal conditions associated with a user of the gas control system over a period of time. The method further includes correlating, at the computer device, changes in the concentration/partial pressure of the second gas and/or changes in the one or more conditions associated with the user with changes in the concentration/partial pressure of the first gas, and calculating, using the computing device, an amount of the first gas or a third gas to be delivered or regulated to the user in response to changes in the concentration/partial pressure of the second gas and/or changes in the one or more conditions associated with the user in order to reach the desired target for the concentration/partial pressure of the first gas to be delivered to the user.

In some implementations, the first gas is oxygen and the second gas is carbon dioxide. In some implementations, the method further includes regulating the amount of the first gas to be delivered to the user using one or more electronically or mechanically actuated valves by oxygen addition, chamber air addition, and/or exhalation air purging. In some implementations, regulating the amount of the first gas comprises controlling the amount of the first gas delivered to the user according to a treatment schedule. In some implementations, the one or more conditions associated with the user comprises at least one of: internal pressure, ambient pressure, volume, temperature, and relative humidity. In some implementations, the desired target for the concentration/partial pressure of the first gas is reached in response to changes in an ambient pressure and/or internal pressure. In some implementations, receiving the data comprises receiving the data from a sensor assembly comprising a plurality of sensors configured to reliably operate even at pressures up to about 3 atmospheres absolute.

### BRIEF DESCRIPTION OF THE DRAWINGS

FIG. 1 shows a schematic illustration of a patient on a mechanical ventilator supplying oxygen-enriched air to the patient.

FIG. 2 shows a schematic illustration of a diver equipped with an oxygen rebreather apparatus.

FIG. 3 shows a schematic illustration of divers undergoing decompression cycles in a submarine decompression chamber or pressurized rescue module.

FIG. 4 shows a schematic block diagram of an example oxygen delivery and reclamation system according to some implementations.

FIG. 5 shows a schematic block diagram of an example multi-person oxygen delivery and reclamation system according to some implementations.

FIG. 6A shows a schematic illustration of an example rebreather device configured to be worn by a user according to some implementations.

FIG. 6B shows a flow diagram of example breathing loop for the rebreather device of FIG. 6A according to some implementations.

FIG. 7 shows a schematic illustration of multiple divers using example rebreather devices coupled to a multi-person oxygen delivery and reclamation system in a submarine decompression chamber according to some implementations.

Like reference numbers and designations in the various drawings indicate like elements.

### DETAILED DESCRIPTION

Several different techniques exist for delivery of oxygen to a user: (1) open circuit, (2) closed circuit, (3) semi-closed circuit, and (4) ambient air combined with supplemental oxygen. In an open circuit device, a user inhales gas (e.g., 100% oxygen) directly from a supply tank or reservoir, and exhaled gas is released to the atmosphere. Oxygen supplies can be quickly consumed in open circuit devices. In a closed circuit device, expired oxygen is scrubbed and recirculated for breathing. A typical closed circuit rebreather includes a breathing bag, a carbon dioxide scrubber, an oxygen source, and connecting hoses. Oxygen is inhaled from the breathing bag, and exhalation gas is scrubbed of carbon dioxide so that remaining oxygen is recirculated back to the breathing bag. Oxygen supplies are consumed less quickly compared to open circuit devices, but closed circuit devices may require other consumables including a chemical scrubber and are generally more complex to operate. Semi-closed circuit devices expel a portion of gas from a breathing loop at various intervals. Typically, supply gas in a semi-closed circuit device is a breathable mixture containing oxygen and a diluent. As oxygen percentage in the breathing loop decreases, more of the breathable mixture is added to the breathing loop. With ambient air combined with supplemental oxygen, a constant flow of oxygen is inhaled simultaneously with air from a surrounding environment.

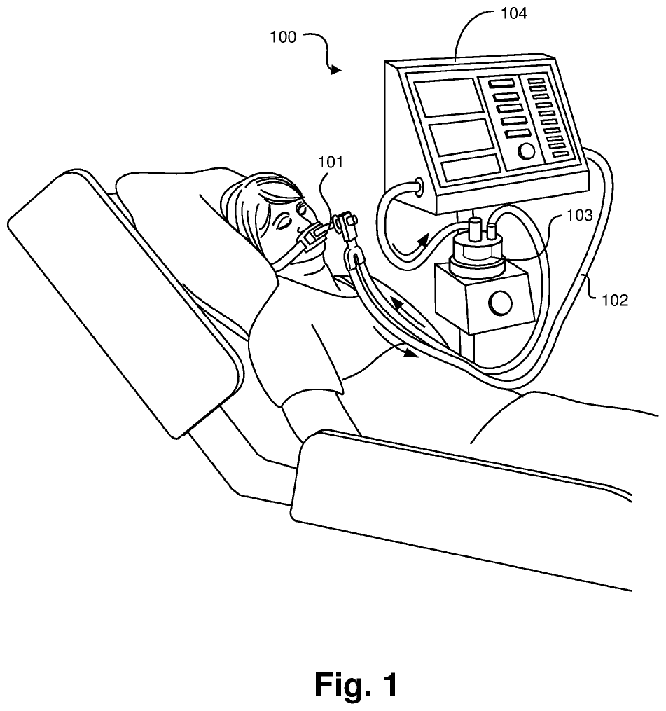

In many hospitals and healthcare environments, oxygen supplies may be consumed by patients experiencing respiratory-related ailments. In some instances, mechanical ventilators may supply oxygen-enriched air (or just air) to a patient. Many mechanical ventilators are ordinarily open circuit devices. FIG. 1 shows a schematic illustration of a patient on a mechanical ventilator supplying oxygen-enriched air to the patient. A mechanical ventilator 100 includes tubes 101 that deliver the oxygen-enriched air into the patient's nose and/or mouth, connecting hoses 102, a humidifier 103 that warms and moistens the oxygen-enriched air, and a control unit 104. The control unit 104 may include various electronics, sensors, valves, and oxygen supply source. Depending on the patient's heart rate, respiratory rate, blood pressure, and/or oxygen saturation in the bloodstream, the control unit 104 may regulate how much oxygen is delivered to the patient. The mechanical ventilator 100 pushes oxygen-enriched air from the control unit 104 through the connecting hoses 102 and the tubes 101 to fill the patient's lungs with the oxygen-enriched air. Exhaled air that contains carbon dioxide is returned back to the control unit 104 to be released via an exhaust port (not shown).

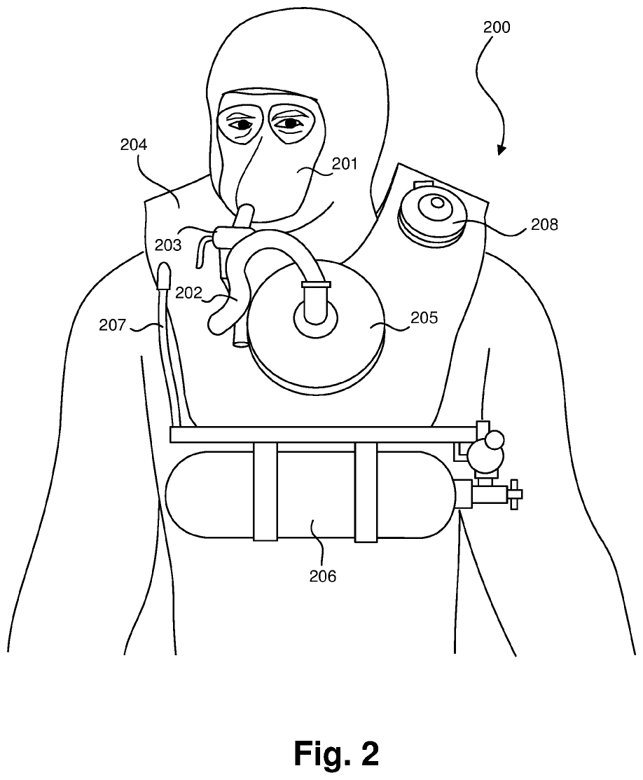

In underwater diving applications, diving rebreathers absorb carbon dioxide from a diver's breath to permit recycling of unused oxygen content, where oxygen is replenished in a breathing loop by at least an amount metabolized by the diver. Unlike open circuit devices that release exhaled breath directly to the environment, many diving rebreathers are closed circuit or semi-closed circuit devices. FIG. 2 shows a schematic illustration of a diver equipped with an oxygen rebreather apparatus. The oxygen rebreather apparatus 200 is a self-contained breathing apparatus that serves to recycle breathing gas by absorbing carbon dioxide and replenishing oxygen metabolized by a diver. The oxygen rebreather apparatus 200 may include a facemask 201 that covers at least a diver's nose and mouth. The facemask 201 may include a mouthpiece (not shown) through which the diver breathes. The mouthpiece may be connected to the rest of the oxygen rebreather apparatus 200 by breathing tubes 202. A dive valve 203 at the mouthpiece may allow the diver to switch breathing between a breathing loop and ambient surroundings. The breathing tubes 202 may be connected to counter-lungs or breathing bag 204. The breathing bag 204 allows the breathing loop to expand and contract when the diver breathes, thereby allowing a fixed closed total volume in the breathing loop. Air may be pushed through the oxygen rebreather apparatus 200 by exhalation. The exhaled air goes through a carbon dioxide removal unit 205 that may include a chemical sorbent for removing carbon dioxide. The exhaled air enters the breathing bag that accommodates the change in volume. The oxygen rebreather apparatus 200 further includes an oxygen supply tank 206 that supplies oxygen into the breathing loop and replenishes the exhaled air with oxygen. The oxygen replenishes the exhaled air via an oxygen feed 207, and the replenished air is pushed back into the breathing bag 204 during inhalation. The oxygen rebreather apparatus 200 may further include an exhaust valve 208 to release breathing gas from the breathing bag 204 to ambient surroundings.

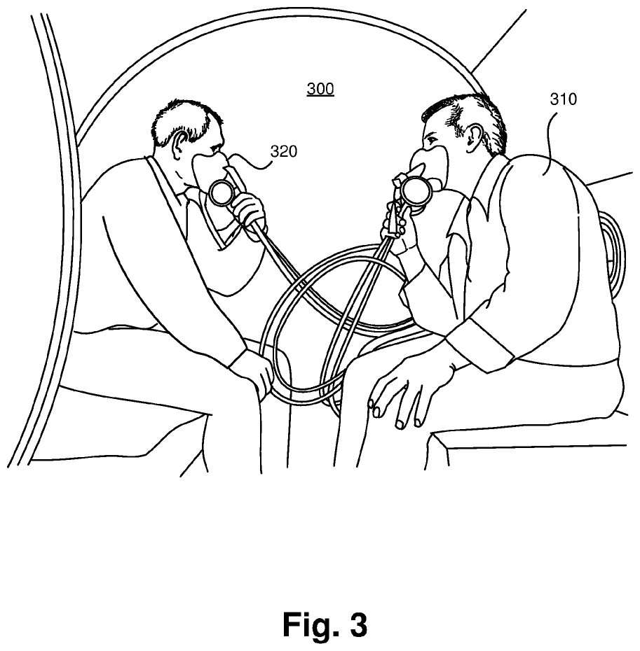

Divers or submariners that are underwater and exposed to elevated pressures for long periods of time may need to undergo a procedure called surface decompression. During surface decompression, individuals generally undergo decompression in a chamber instead of in water. The chamber provides a controlled environment where oxygen can be delivered to each of the individuals at greater partial pressures. FIG. 3 shows a schematic illustration of divers undergoing decompression cycles in a submarine decompression chamber or pressurized rescue module. By way of illustration, trapped underwater submariners or divers 310 may be transferred to a pressurized rescue module (PRM) having a submarine decompression chamber (SDC) 300 prior to returning to the surface. The submarine decompression chamber 300 may hold several personnel, such as 18 to 35 submariners or divers 310 at a time. The submariners or divers 310 in the submarine decompression chamber 300 receive oxygen or oxygen-enriched air according to a certain schedule. The oxygen or oxygen-enriched air may be received via personalized breathing apparatuses 320. Higher levels of oxygen may be delivered to increase the rate of decompression. In some cases, decompression treatment can take several hours and even days. After decompression treatment, chamber pressure is gradually reduced in the submarine decompression chamber 300.

The present disclosure relates to an oxygen delivery and reclamation system for automated delivery of oxygen-enriched gas to one or more users. The oxygen delivery and reclamation system may serve multiple users. Rather than individualized oxygen supplies, the oxygen delivery and reclamation system may share a central oxygen supply for multiple users. The oxygen delivery and reclamation system may reduce decompression time. Furthermore, the oxygen delivery and reclamation system reduces net oxygen consumption, thus reducing consumables that can be quickly depleted in many oxygen delivery systems. Gas lines are minimized in the oxygen delivery and reclamation system by utilizing chamber air instead of a separate diluent source. Though the oxygen delivery and reclamation system may have a central oxygen supply for multiple users, each user may have a rebreather so that breathing gas flow may be decoupled from the central oxygen supply and each other. This permits individual control of oxygen levels to various users, reduces risk of system-wide contamination, and reduces work of breath in circulating breathing gas. Exhaled gas may be exhausted or purged from rebreathers using vacuum lines and/or valves. This is done in a controlled and automated manner without assistance from the user's lungs. Recycling of oxygen may occur by collecting the exhaled gas and/or chamber air with an oxygen concentrator in the oxygen delivery and reclamation system.

Many open circuit devices (e.g., FIG. 1) and some closed circuit devices (e.g., FIG. 2) consume oxygen from individualized oxygen sources. Oxygen supplies and even carbon dioxide scrubbers are consumables that can be depleted quickly in many oxygen delivery systems (e.g., FIGS. 1-3). Medical, marine, aerospace, mining, or other settings may have limited oxygen supplies. The oxygen delivery and reclamation system of the present disclosure can recycle oxygen across multiple users to reduce oxygen consumption of oxygen supplies.

Typical oxygen delivery systems either deliver oxygen to a single user from a single oxygen source (e.g., FIGS. 1 and 2) or deliver oxygen to multiple users from a central oxygen source (e.g., FIG. 3). Generally, centralized oxygen delivery systems do not provide oxygen levels specific to individual users. For instance, several submariners undergoing decompression may receive inhalation gas at 100% oxygen. With the assistance of various controllers, sensors, electronics, valves, and individualized rebreathers, however, the oxygen delivery and reclamation system may provide different levels of oxygen for different users. Thus, as some users may be more sensitive to higher levels of oxygen than others, the oxygen delivery and reclamation system may control how much oxygen is delivered to each user.

Many decompression chambers (e.g., FIG. 3) and semi-closed circuit devices use separate diluent sources to reduce oxygen or carbon dioxide levels. Separate diluent sources require additional gas sources such as inert gas supplies, not to mention additional gas lines and controls. This can add to volume, mass, cost, and complexity to an oxygen delivery system. The oxygen delivery and reclamation system of the present disclosure utilizes ambient chamber air to supply diluent for recycling oxygen and controlling oxygen and carbon dioxide levels. Utilizing ambient chamber air also maintains a constant or substantially constant volume in the oxygen delivery and reclamation system so that there is negligible volume change.

The oxygen delivery and reclamation system of the present disclosure uses an oxygen concentrator that receives one or both of exhalation gas and chamber air as input, and releases oxygen-enriched gas and oxygen-depleted gas as output. Instead of evacuating exhalation gas into atmosphere or other ambient environment, the oxygen delivery and reclamation system recycles oxygen by converting exhalation gas and/or chamber air into high-purity oxygen. Ordinarily, oxygen delivery systems (e.g., FIGS. 1-3) that recycle oxygen require a carbon dioxide scrubber. However, the oxygen delivery and reclamation system of the present disclosure may continue to operate in a semi-closed circuit mode even without a carbon dioxide scrubber and/or oxygen supply source. Though the oxygen delivery and reclamation system of the present disclosure may be shown with a carbon dioxide scrubber and/or oxygen supply source, it will be understood that such components may not be necessary for successful oxygen delivery and reclamation.

Generally speaking, oxygen delivery systems that recycle oxygen often require user(s) to push gas by exhalation to circulate the gas. The user exerts effort to move breathing gas through a system so that the system depends on the user's skill and lung functionality to purge and circulate breathing gas. The oxygen delivery and reclamation system of the present disclosure employs controlled purge lines and electronically actuated gas delivery lines for quick and low-effort or effortless delivery of oxygen-enriched gas. That way, the oxygen delivery and reclamation system does not depend on the user's lungs to circulate gas. Instead, gas can be moved/removed from the breathing loop in a controlled and automated manner without relying on the user's lungs or blowers to push gas. Even if a user's lungs were medically impaired, for example, the oxygen delivery and reclamation system can continue to circulate breathing gas and recycle oxygen.

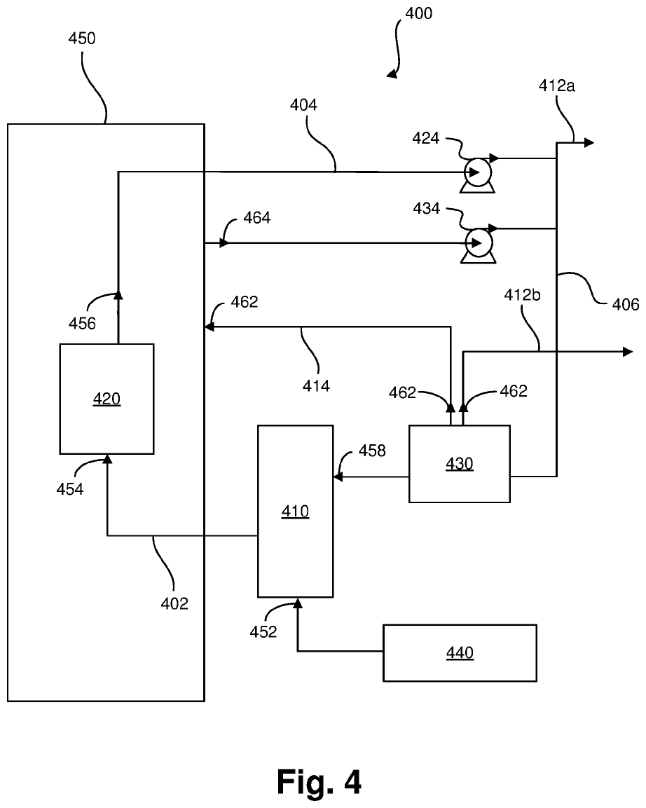

FIG. 4 shows a schematic block diagram of an example oxygen delivery and reclamation system according to some implementations. The oxygen delivery and reclamation system 400 includes an oxygen-enriched gas supply source 410, a chamber 450, a rebreather device 420 in the chamber 450, a purge line 404 fluidly coupled to the rebreather device 420, a recycle line 406 fluidly coupled to the purge line 404, and an oxygen concentrator 430. The oxygen concentrator 430 may be fluidly coupled to the oxygen-enriched gas supply source 410, where the oxygen concentrator 430 provides oxygen-enriched gas to the oxygen-enriched gas supply source 410. In some implementations, an oxygen tank 440 may also be fluidly coupled to the oxygen-enriched gas supply source 410 to provide oxygen (i.e., 100% or high-concentration oxygen) to the oxygen-enriched gas supply source 410. It will be understood that the oxygen from the oxygen tank 440 may not necessarily be 100% oxygen, but may be substantially pure oxygen that is at least 95% oxygen, at least 98% oxygen, or at least 99% oxygen. A gas delivery line 402 may be fluidly coupled to the rebreather device 420 and provide oxygen-concentrated inhalation gas from the oxygen-enriched gas supply source 410 to the rebreather device 420. A pressure-regulated mechanism such as a valve, regulator, or variable restrictor may purge exhalation gas from the purge line 404 to a recycle line 406. A pressure-regulated mechanism such as a valve, regulator, or variable restrictor may supply chamber air from the chamber 450 to the recycle line 406. The exhalation gas, chamber air, or a mixture thereof may be flowed through the recycle line 406 to the oxygen concentrator 430.

The oxygen delivery and reclamation system 400 may be contained in an enclosed space such as the chamber 450. The chamber 450 may be a vessel such as a pressurized vessel. Examples of pressurized vessels may be submarine decompression chambers or pressurized rescue modules. The oxygen-enriched gas supply source 410, the rebreather device 420, the purge line 404, the recycle line 406, the oxygen concentrator 430, and the oxygen tank 440 may be located in the chamber 450. However, it will be understood that in certain embodiments one or more of the oxygen-enriched gas supply source 410, the rebreather device 420, the purge line 404, the recycle line 406, the oxygen concentrator 430, and the oxygen tank 440 may be located outside the chamber 450.

In FIG. 4, straight-lined arrows indicate the flow of gas that circulates in the oxygen delivery and reclamation system 400. Oxygen-concentrated inhalation gas 454 is supplied via the gas delivery line 402 from the oxygen-enriched gas supply source 410 to a user of the rebreather device 420. The oxygen-concentrated inhalation gas 454 is supplied to the rebreather device 420 as needed. It is not a constant feed. In some embodiments, activation of a valve and controller coupled to the rebreather device 420 may control delivery of the oxygen-concentrated inhalation gas 454 to the user. Depending on a desired oxygen concentration, an oxygen level setpoint may be established for a particular user of the rebreather device 420. The oxygen level setpoint may be user-specified or provided from a control station. A controller may receive an indication of a desired oxygen level to be supplied to a user of a rebreather device 420. The oxygen level setpoint may depend on various factors such as the user's height, weight, age, breathing capacity, medical needs, decompression schedule, etc. In some embodiments, the oxygen level setpoint may be between 21% oxygen by volume and about 100% oxygen by volume. For example, the oxygen-concentrated inhalation gas 454 may be at least 90% oxygen by volume. In some embodiments, the oxygen-concentrated inhalation gas 454 may be a mixture of oxygen and nitrogen or a mixture of oxygen, nitrogen, and water vapor. The oxygen-concentrated inhalation gas 454 may be diluted with chamber air from the chamber 450. This allows the user to be treated with the desired oxygen concentration.

The rebreather device 420 provides a breathing loop that receives the oxygen-concentrated inhalation gas 454 and releases exhalation gas 456. Exhalation gas 456 is exhaled by the user that generally contains more carbon dioxide and water vapor and less oxygen than the oxygen-concentrated inhalation gas 454. The exhalation gas 456 may also be referred to as exhaled breath, exhaled air, expired gas, breathing gas, and purge gas. In some embodiments, the exhalation gas 456 is replenished of oxygen metabolized by the user within the breathing loop, where the exhalation gas 456 is scrubbed by a carbon dioxide scrubber in the rebreather device 420 to provide breathing gas to the user. In some embodiments, the exhalation air 456 is recycled of oxygen by purging the exhalation gas 456 out of the breathing loop. The exhalation gas 456 is collected in the purge line 404, where the purge line 404 is connected to the breathing loop or rebreather device 420. The exhalation gas 456 collected in the purge line 404 may be purged in an automated and controlled manner with or without assistance from the user's lungs. The exhalation gas 456 may be either purged to the recycle line 406 or vented out to atmosphere or an environment outside the chamber 450 in a first atmospheric line 412a. A purge pump 424 connected to the purge line 404 may be actuated to pull the exhalation gas 456 to the recycle line 406 or the first atmospheric line 412a. The purge pump 424 may pull a vacuum on the purge line 404 to purge the exhalation gas 456 from the breathing loop. Upon actuation, a pressure differential or vacuum is generated in the recycle line 406 to pull the exhalation gas 456 from the purge line 404 to the recycle line 406, or a pressure differential or vacuum is generated in the first atmospheric line 412a to pull out the exhalation gas 456 to the atmosphere or the environment outside the chamber 450. If recycling of the exhalation gas 456 is not needed, the exhalation gas 456 may be vented to the atmosphere via the first atmospheric line 412a. As a result, the exhalation gas 456 may be circulated for oxygen reclamation or vented even without user effort. In contrast, many conventional oxygen delivery systems that operate in a closed loop often require exertion from the user's lungs to circulate gas.

The exhalation gas 456 accumulates in and passes through the recycle line 406 and feeds into the oxygen concentrator 430. The recycle line 406 is fluidly coupled to the oxygen concentrator 430. In some implementations, a chemical scrubber or chemical sorbent such as a carbon dioxide scrubber (not shown) is upstream of the oxygen concentrator 430. The chemical sorbent may remove a chemical such as carbon dioxide from the exhalation gas 456. In some embodiments, the chemical sorbent may release heat and water vapor as a byproduct of removal of carbon dioxide, thereby heating and humidifying the exhalation gas 456. The exhalation gas 456 is received by the oxygen concentrator 430, where the oxygen concentrator 430 receives low-purity oxygen and outputs high-purity oxygen. In some implementations, the oxygen concentrator 430 may be an electrochemical oxygen concentrator, a vacuum swing adsorption (VSA) oxygen concentrator, or a pressure swing adsorption (PSA) oxygen concentrator.

In some embodiments, the oxygen concentrator 430 receives a feed of air such as the exhalation gas 456 and outputs oxygen-enriched gas 458 and oxygen-depleted gas 462. The oxygen concentrator 430 may output the oxygen-enriched gas 458 to the oxygen-enriched gas supply source 410 and output the oxygen-depleted gas 462 to one of the following: the chamber 450 via a chamber line 414, the environment outside the chamber 450 via a second atmospheric line 412b, or a storage source (not shown). The oxygen-enriched gas 458 may be at least about 70% oxygen by volume, at least about 80% oxygen by volume, or at least about 90% oxygen by volume. The oxygen-depleted gas 462 may contain less than about 25% oxygen by volume, less than about 22% oxygen by volume, or between about 17% and about 22% oxygen by volume. The oxygen-depleted gas 462 may contain oxygen balanced by nitrogen. The oxygen-depleted gas 462 may also be referred to as normoxic air.

Additionally or alternatively, chamber air 464 may be fed from the chamber 450 into the recycle line 406. Typically, chamber air 464 includes ambient air located in the vessel and surrounding the user of the rebreather device 420. Chamber air 464 may contain less than about 25% oxygen by volume, less than about 22% oxygen by volume, or between about 17% and about 22% oxygen by volume. The chamber air 464 may be pulled into the recycle line 406 by a pressure differential or vacuum. In some implementations, pulling the chamber air 464 into the recycle line 406 may be assisted by a chamber pump 434. The chamber pump 434 may regulate the amount of the chamber air 464 being pulled into the recycle line 406. Pulling chamber air 464 from the chamber 450 may serve to maintain a constant volume in the chamber 450 as a result of mask leakage. The chamber air 464 may feed from the recycle line 406 into the oxygen concentrator 430. In some embodiments, a chemical sorbent upstream of the oxygen concentrator 430 may remove carbon dioxide from the chamber air 464. The oxygen concentrator 430 receives a feed of air such as the chamber air 464 and outputs oxygen-enriched gas 458 and oxygen-depleted gas 462. In some implementations, the chamber air 464 is combined with the exhalation gas 456 to form a mixture of the chamber air 464 and the exhalation gas 456 that feeds into the oxygen concentrator 430. In some other implementations, the chamber air 464 and the exhalation gas 456 are fed separately into the oxygen concentrator 430.

In some implementations, an oxygen tank 440 is fluidly coupled to the oxygen-enriched gas supply source 410. The oxygen tank 440 provides oxygen 452 (i.e., 100% oxygen or high-concentration oxygen) to the oxygen-enriched gas supply source 410. In case there is an issue with the recycling of oxygen in the recycle line 406 and/or the oxygen concentrator 430, the oxygen tank 440 may ensure that there is oxygen 452 being fed to the gas delivery line 402. Thus, the oxygen 452 from the oxygen tank 440 may provide oxygen to the oxygen-enriched gas supply source 410. Or, the oxygen 452 from the oxygen tank 440 may supplement the oxygen-enriched gas 458 from the oxygen concentrator 430 to form a more oxygen-concentrated gas mixture in the oxygen-enriched gas supply source 410. The oxygen-enriched gas 458, the pure oxygen 452, or the more oxygen-concentrated gas mixture that combines the oxygen-enriched gas 458 and the oxygen 452 provides the gas source for the oxygen-concentrated inhalation gas 454. Even without the oxygen tank 440 providing oxygen 452 to the oxygen-enriched gas supply source 410, the oxygen delivery and reclamation system 400 may operate in a closed or semi-closed loop.

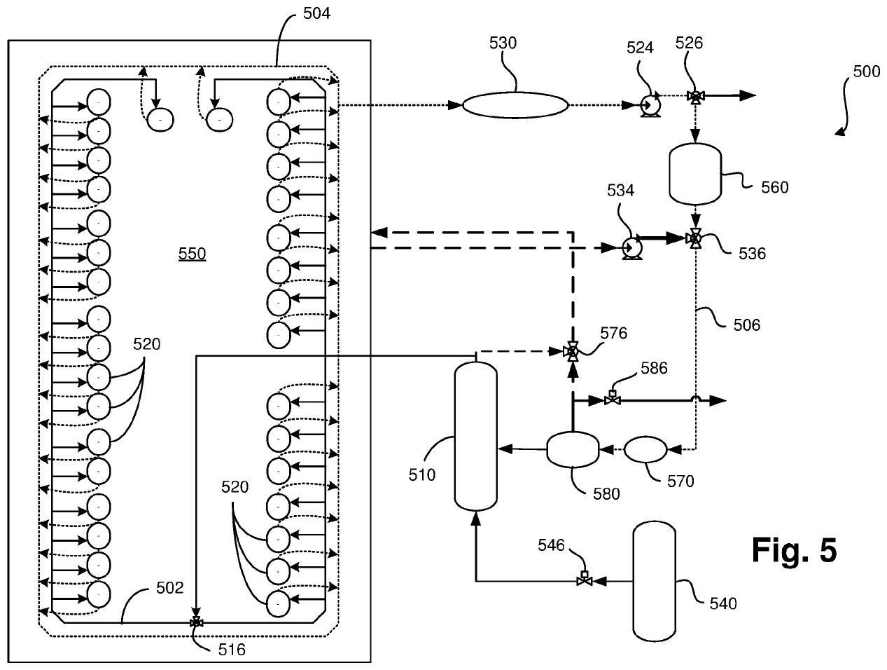

FIG. 5 shows a schematic block diagram of an example multi-person oxygen delivery and reclamation system according to some implementations. The oxygen delivery and reclamation system 500 includes an oxygen-enriched gas supply source 510, a chamber 550, a plurality of rebreather devices 520 adapted for a plurality of users (e.g., divers) in the chamber 550, one or more gas delivery lines 502, one or more purge lines 504, a recycle line 506, and an oxygen concentrator 580. Each of the plurality of rebreather devices 520 comprises a mask configured to be worn by a user. The one or more gas delivery lines 502 are fluidly coupled to inlets of the plurality of rebreather devices 520 for receiving inhalation gas. The one or more purge lines 504 are fluidly coupled to outlets of the plurality of rebreather devices 520 for discharging exhalation air. The one or more purge lines 504 may be fluidly coupled to the recycle line 506 via one or both of a recycle pump 524 and an electronically actuated valve 526. The oxygen concentrator 580 may be fluidly coupled to the recycle line 506 for receiving one or both of exhalation air and chamber air. Chamber air may be pulled into the recycle line 506 via one or both of a recycle pump 534 and an electronically actuated valve 536. The oxygen concentrator 580 may convert air (i.e., exhalation air and/or chamber air) received from the recycle line 506 into oxygen-enriched gas and oxygen-depleted gas. The oxygen-enriched gas may be supplied to the oxygen-enriched gas supply source 510. The oxygen-enriched gas supply source 510 may provide desired oxygen levels to each of plurality of users via the one or more gas delivery lines 502.

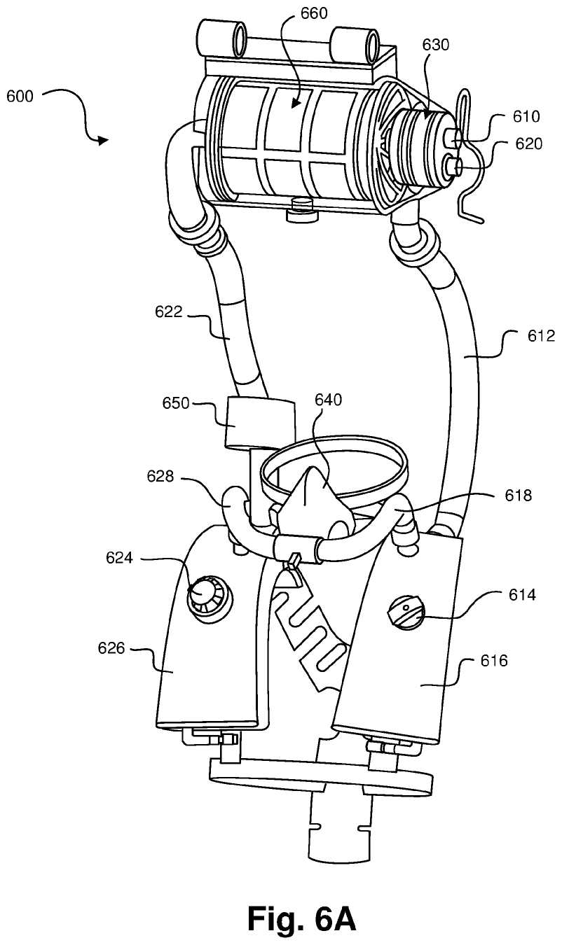

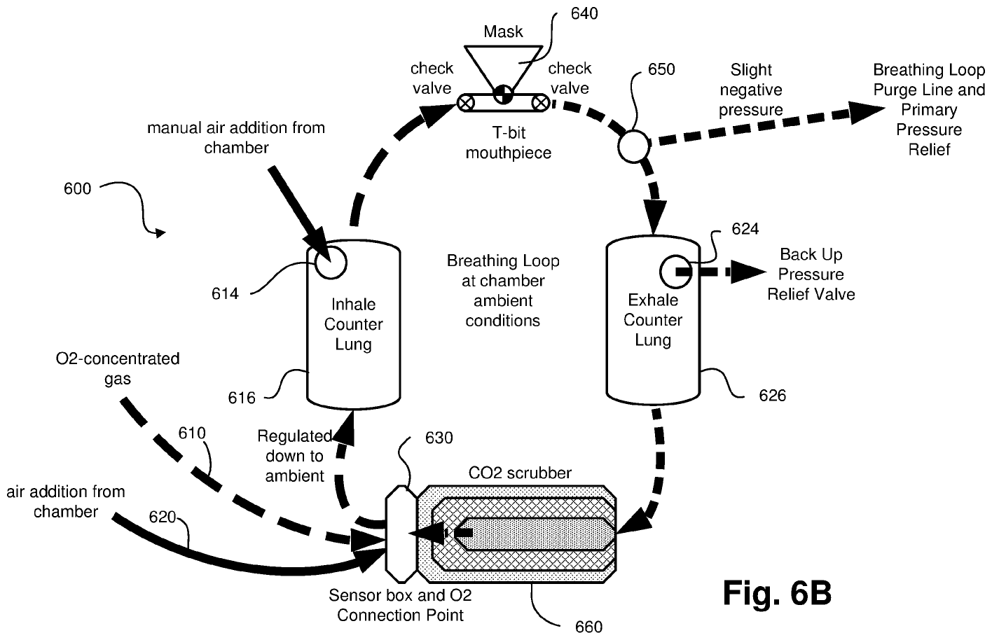

The multi-person oxygen delivery and reclamation system 500 includes a plurality of rebreather devices 520. A rebreather device 520 may also be referred to as a “rebreather,” “rebreather loop,” or “breathing loop.” Each rebreather device 520 is equipped with an intake line or inlet for receiving oxygen-enriched gas from the one or more gas delivery lines 502 and is equipped with an outtake line or outlet for discharging exhalation air to one or more purge lines 504. The rebreather device 520 facilitates recycling of oxygen without forcing exhalation air to be discharged directly to an ambient environment. The rebreather device 520 may also be equipped with an oral nasal mask fitted over the mouth and nose of each user. The rebreather device 520 is connected to the oxygen delivery and reclamation system 500 at a gas delivery line 502 for oxygen or oxygen-enriched gas intake and at a purge line 504 for purging exhalation air. Aspects of the rebreather device 520 are described in further detail below with respect to FIGS. 6A and 6B.

FIG. 6A shows a schematic illustration of an example rebreather device configured to be worn by a user according to some implementations. FIG. 6B shows a flow diagram of an example breathing loop for the rebreather device of FIG. 6A according to some implementations. As shown in FIGS. 6A and 6B, a rebreather device 600 can include an oxygen intake valve 610, a chamber air intake valve 620, a sensor assembly 630, an oral nasal mask 640, a purge valve 650, and a carbon dioxide scrubber 660. The rebreather device 600 further includes two breathing bags 616, 626 that undergo a change in volume to accommodate inhalation and exhalation by the person, thereby allowing a fixed closed total volume within a breathing loop. Each of the breathing bags 616, 626 include relief valves 614, 624 that open up to an ambient environment in case the breathing loop becomes over-pressurized. The breathing bags 616, 626 may also be referred to as “counter lungs” or “differential volumes.” Each of the breathing bags 616, 626 may be worn by a user.

An inhale breathing bag 616 is configured to receive oxygen-enriched gas via an oxygen tube 612. The oxygen-enriched gas may come from various sources. As shown in FIGS. 6A and 6B, one source may be pure oxygen or oxygen-concentrated gas introduced via the oxygen intake valve 610. In some embodiments, the oxygen intake valve 610 may be electronically actuated, where a controller is configured to automatically open/close the oxygen intake valve 610. In some embodiments, the oxygen intake valve 610 includes a solenoid. The oxygen intake valve 610 may receive pure oxygen or oxygen-concentrated gas (e.g., nitrox) from an oxygen-enriched gas supply source that may be delivered from a centralized oxygen supply in an oxygen delivery and reclamation system as shown in FIGS. 4 and 5. In FIG. 5, an electronically or mechanically actuated valve 516 delivers oxygen-enriched gas to a system 500 and the oxygen intake valve 610 in FIG. 6 can regulate the oxygen-concentrated gas to individual rebreather devices 600/520. Another source may be chamber air introduced via the chamber air intake valve 620. In some embodiments, the chamber air intake valve 620 may be electronically actuated, where a controller is configured to automatically open/close the chamber air intake valve 620. The controller may be configured to control a composition of gas being introduced into the rebreather device 600 and aids with regulation of a partial pressure of oxygen in the chamber. In some embodiments, the controller and sensor(s) may be housed in the sensor assembly 630 or located in the chamber (e.g., decompression chamber). In some embodiments, the chamber air intake valve 620 includes a solenoid. The chamber air may be pulled from an ambient environment such as a pressurized vessel. The chamber air may act as a diluent to the pure oxygen or oxygen-concentrated gas. Additionally or alternatively, chamber air may be introduced into the inhale breathing bag 616 manually via a chamber air addition valve 614. The chamber air addition valve 614 may serve as an anti-suffocation valve that will open to the ambient environment (e.g., chamber) if the breathing loop does not contain enough oxygen-enriched gas for breathing. Another source may be scrubbed exhalation air introduced via the carbon dioxide scrubber 660. Exhalation air that is scrubbed of carbon dioxide may be recycled for inhalation. The scrubbed exhalation air may provide heated and humidified air to the oxygen-enriched gas. The pure oxygen or oxygen-concentrated gas, the chamber air, the scrubbed exhalation air, or combinations thereof may be supplied to the inhale breathing bag 616 via the oxygen tube 612.

The inhale breathing bag 616 provides an enclosed volume to accommodate inhalation. The oxygen-enriched gas flows from the inhale breathing bag 616 to an inlet of the oral nasal mask 640. The inlet of the oral nasal mask 640 is fluidly connected to the inhale breathing bag 616 via an inlet tube 618. The oral nasal mask 640 may be adapted to engage a person's face so that an enclosed space is formed over the person's mouth and nasal openings. The inlet of the oral nasal mask 640 receives the oxygen-enriched gas and an outlet of the oral nasal mask 640 receives exhalation air breathed out by a user of the oral nasal mask 640. In some embodiments, the oral nasal mask 640 includes a mouthpiece (e.g., T-bit mouthpiece) with one or more one-way check valves to ensure airflow proceeds in one direction. The exhalation air is discharged from the outlet of the oral nasal mask 640 and into an exhale breathing bag 626 via an outlet tube 628. The oral nasal mask 640 may be designed to minimize leakage into the ambient environment.

An exhale breathing bag 626 is configured to receive exhalation air via the outlet tube 628. The exhalation air contains more carbon dioxide and less oxygen than the oxygen-enriched gas. The exhale breathing bag 626 provides an enclosed volume to accommodate exhalation. As exhalation air accumulates in the exhale breathing bag 626, some of the exhalation air is pushed through a carbon dioxide tube 622 to a carbon dioxide scrubber 660 for removing carbon dioxide. In other words, some of the exhalation air is pushed through the breathing loop for recycling breathable gas (scrubbed exhalation air) to the user. This provides a closed-loop airflow system in the rebreather device 600.

Cycling exhalation air through the carbon dioxide tube 622 to the carbon dioxide scrubber 660 is one flow path for recycling breathable gas back to the user. Another flow path for recycling breathable gas back to the user involves purging the exhalation air via a purge valve 650 that may be mechanically, electronically, or manually actuated. This takes the exhalation air out of the breathing loop. In some embodiments, the purge valve 650 may be electronically actuated, where a controller is configured to automatically open/close the purge valve 650. As more exhalation air accumulates in the breathing loop, the purge valve 650 may open to discharge the exhalation air for oxygen reclamation using an oxygen delivery and reclamation system as described in FIGS. 4 and 5. In particular, the exhalation air can be discharged or pumped out to a recycle line (e.g., recycle line 406 in FIG. 4 or recycle line 506 in FIG. 5), where the recycle line pulls the exhalation air by vacuum or pressure differential. Oxygen can be replenished using an oxygen concentrator. The oxygen concentrator receives the purged exhalation air and converts it to oxygen-enriched air, which can then be fed back to the user as oxygen-concentrated gas through the oxygen intake valve 610. Thus, the rebreather device 600 is connected to a centralized oxygen delivery and reclamation system at the purge valve 650 for purging exhalation air and at the oxygen intake valve 610 for introducing pure oxygen or oxygen-concentrated gas. Purging the exhalation air from the breathing loop allows the rebreather device 600 to be purged quickly with no or minimal user interaction or effort and independently of lung capacity, lung strength, and breathing rate so that a higher oxygen percentage can be brought into the breathing loop faster than if the rebreather device 600 lacked a purge mechanism. Specifically, purging of the exhalation air from the breathing loop can occur with little correlation to user lung capacity, lung strength, and breathing rate. Normally, exhalation air can be cycled through the carbon dioxide scrubber 660. However, the purge valve 650 may regulate over-pressurization and also purge the breathing loop to get the oxygen concentration to a desired setpoint more rapidly. Exhalation air rich in carbon dioxide may be purged for oxygen reclamation according to a schedule, in case of scrubber failure, in case of an increase in bag volume due to over-pressurization, user actuation, or other reason.

The sensor assembly 630 in the rebreather device 600 may include one or more sensors. The sensor assembly 630 provides accurate and automated monitoring of various conditions in the breathing loop (or outside the breathing loop, e.g., ambient pressure). In some implementations, the sensor assembly 630 monitors oxygen levels, carbon dioxide levels, trace gas levels, pressure, temperature, relative humidity, volume, or combinations thereof. Oxygen levels, carbon dioxide levels, and other gas levels (e.g., nitrogen levels) may be monitored by measuring partial pressures. Example oxygen sensor types include but are not limited to galvanic sensors, electrochemical sensors, optical laser-tuned diode sensors, LED optical sensors, paramagnetic oxygen sensors, fluorescent sequencing-luminescence sensors, and zirconia sensors. Example carbon dioxide sensor types include but are not limited to optical laser-tuned diode sensors, non-dispersion infrared sensors, fluorescent sequencing-luminescence sensors, and electrochemical sensors. In addition to oxygen and carbon dioxide sensors and sensors for trace gases, the sensor assembly 630 may further include pressure sensors, temperature sensors, relative humidity sensors, or combinations thereof. One or more of the oxygen sensors, carbon dioxide sensors, trace gas sensors, pressure sensors, temperature sensors, and relative humidity sensors may be enclosed in a housing of the sensor assembly 630. In some embodiments, the sensors in the sensor assembly may be configured to reliably operate even at pressures up to about 3 atmospheres absolute (ATA). Data regarding oxygen partial pressure (PPO₂), carbon dioxide partial pressure (PPCO₂), rebreather internal pressure, ambient pressure, volume, temperature, and relative humidity is measured and received by a controller or electronic control system.

The sensor assembly 630 provides for accurate monitoring of the gas stream in the breathing loop. The sensor assembly 630 measures conditions in the breathing loop such as PPO₂, PPCO₂, rebreather internal pressure, ambient pressure, volume, temperature, and relative humidity. Other conditions may be measured and other quantities may be calculated. These measurements and calculations may be stored, logged, recorded, and processed by an electronic control system or controller. The controller may include at least one of a general purpose single- or multi-chip processor, a digital signal processor (DSP), an application specific integrated circuit (ASIC), a field programmable gate array (FPGA), or other programmable logic device, discrete gate or transistor logic, or discrete hardware components. The controller may be configured to control the operations of the rebreather device 600 according to instructions (e.g., software) stored on one or more non-transitory computer-readable media. Such non-transitory media may include a memory such as volatile memory, non-volatile memory, or combinations thereof. The controller receives data from the sensor assembly 630 and may be configured to ensure that minimum oxygen levels and maximum oxygen levels are not exceeded, thereby preventing undesired or unsafe breathing conditions. Holding safe breathing conditions can prevent oxygen toxicity while enabling appropriate oxygen treatment or decompression. By way of an example, the controller may be configured to ensure minimum oxygen levels of at least 90% oxygen by volume in the breathing loop and maximum oxygen levels of 2.8 ATA oxygen partial pressure (at 100% oxygen by volume). The oxygen levels may be maintained within a desired range irrespective of changes in pressure, temperature, and relative humidity. Where undesired or unsafe conditions are determined, the controller may be configured to provide an alert indicative of the undesired or unsafe conditions. The alert may be provided as visual, auditory, or haptic feedback to one or more users and attendants.

The controller may also receive data from a sensor assembly and may be configured to reach desired setpoints for a first gas concentration or first gas partial pressure. The controller may also be configured to reach desired setpoints for a second gas concentration or second gas partial pressure, a third gas concentration or third gas partial pressure, and so forth. By way of an example, the first gas may be oxygen and the second gas may be carbon dioxide. In some implementations, the third gas may be nitrogen. The controller accounts for changes in conditions associated with the user(s) of a gas control system such as an oxygen delivery and reclaim system. In addition to the controller continually monitoring gas concentrations or partial pressures, the controller may be continually monitoring changes in temperature, internal pressure, ambient pressure, volume, and relative humidity, among other conditions. Changes in any of the aforementioned conditions may affect how much of a first gas, second gas, third gas, etc. gets delivered to a user. The controller may adaptively add certain gas(es) or subtract certain gas(es) from the gas control system based at least in part on changes in any of the aforementioned conditions associated with the user(s). Thus, the controller provides a smart/adaptive gas control architecture with feedback and feedforward control. The controller may provide gas control to one or more specific users of the gas control system rather than all users of the gas control system.

In one example, the controller may measure an oxygen level by measuring PPO₂ associated with a user to be 0.3 PPO₂. If the desired oxygen level setpoint is 0.9 PPO₂, then the controller may be configured to add oxygen to the rebreather device until 0.9 PPO₂ is reached. In another example, the controller may measure an oxygen level by measuring PO₂ associated with a user to be 1.6 PPO₂. If the desired oxygen level setpoint is 0.9 PPO₂, then the controller may be configured to introduce an air break and/or purge the breathing loop. An air break may be introduced for a duration until the desired oxygen level setpoint is reached.

Various factors or conditions associated with the user(s) may be monitored, tracked, logged, and even controlled to influence delivery of a gas (e.g., oxygen) to the user(s). By way of an example, changes in depth or changes in ambient pressure affect the partial pressure of the gas. So to maintain a desired oxygen level setpoint, a depth change may cause the PPO₂ to go too high or too low, causing the controller of the gas control system to add O₂, subtract O₂ (vent/purge), or add air/nitrogen. By way of another example, changes in volume may be indicative of a user's lung capacity, breathing rate (shallow breathing or deep breathing), and/or tidal volume. Moreover, such changes in volume may be associated with changes in oxygen consumption and internal pressure. Specifically, the metabolic consumption of oxygen may be accounted for. Accordingly, the controller may be configured to add O₂, subtract O₂, or add air/nitrogen for controlling PPO₂ to the user(s).

The percentage of oxygen that a person actually inhales is measured by the fraction of inspired oxygen (FiO₂). The controller may be configured to track and control FiO₂, though it will be understood that the controller may alternatively be configured to track and control PPO₂. The actually delivered FiO₂ depends not only on PPO₂ but also on temperature, internal pressure, ambient pressure, volume, and relative humidity. A controller receives and analyzes the data for PPO₂, rebreather internal pressure, ambient pressure, volume, temperature, and relative humidity from the sensor assembly 630. The controller may be configured to calculate FiO₂ based at least in part on the PPO₂, rebreather internal pressure, temperature, and relative humidity measurements. Moreover, the controller may be configured to calculate other metrics or quantities. For instance, the controller may be configured to calculate oxygen concentration or dry-air oxygen concentration using PPO₂. In addition to measurements monitored by the sensor assembly 630, quantities tracked by the controller may further include but are not limited to oxygen consumption, oxygen leakage or loss, breathing rate, depth/pressure change, ambient pressure, volume change, tidal volume, and temperature variation. Accordingly, challenges exist to holding oxygen levels in the breathing loop within specified oxygen level setpoints due to changes in breathing characteristics and changes in the environment such as temperature and pressure changes. Oxygen levels can quickly fluctuate due to changes in rebreather internal pressure, ambient pressure, volume, temperature, humidity, oxygen consumption, breathing rate, mask leakage, and denitrogenation. Using a mathematical model or algorithm that accounts for the aforementioned variables, the controller can determine PPO₂, FiO₂, or dry-air oxygen concentration. PPO₂, FiO₂, or dry-air oxygen concentration can be modeled as conditions in the breathing loop and ambient environment change. That way, FiO₂ levels do not need to be constantly monitored or manually calculated.

The controller may be configured to implement the mathematical model or algorithm to ensure specified oxygen level setpoints are met. In some embodiments, the mathematical model or algorithm can ensure that FiO₂ levels are regulated within a desired range, such as a range between about 90% and about 100% (between about 0.9 and about 1.00). Rather than relying on users to manually adjust oxygen addition as breathing loop conditions and ambient conditions change, the controller may implement the mathematical model or algorithm to appropriately control oxygen addition via oxygen intake valve 610, chamber air addition via chamber air intake valve 620, and exhalation air purging via purge valve 650. This allows for automated oxygen and diluent addition as well as purging by electronically or mechanically actuated valves. Gas composition and levels can be regulated by the controller using the electronically or mechanically actuated valves so that gas mixtures with high- or low-concentration oxygen can be supplied to achieve specified oxygen levels in the breathing loop. The gas composition and levels can be quickly adjusted by the controller even for users with low lung capacity or reduced lung strength. Even if a gas control system were to lose a CO₂ scrubber or the CO₂ scrubber runs out, the controller may operate the gas control system in a semi-closed loop.

In some implementations, the controller may be configured to detect or measure levels of carbon dioxide in the breathing loop. The controller may be configured to purge the breathing loop in response to the detected/measured levels of carbon dioxide. For example, the controller may be configured to purge the breathing loop from exhalation air rich in carbon dioxide every few breaths depending on a user's breathing rate. In some implementations, the controller may be configured to detect or measure high levels of oxygen in the breathing loop that is caused by increased depth. In such circumstances, the controller may be configured to reduce levels of oxygen by opening a purge valve (e.g., purge valve 650) or a chamber relief valve (e.g., relief valve 614 or 624)

Integration of the controller for controlling gas composition and levels by oxygen addition, chamber air addition, and exhalation air purging with monitoring of conditions such as PPO2, PPCO2, internal pressure, ambient pressure, volume, temperature, and relative humidity can provide an adaptive breathing gas control architecture for each user of the rebreather device 600. The controller may receive feedback from the sensor assembly 630 and provide feedforward control to the rebreather device 600 at the oxygen intake valve 610, the chamber air intake valve 620, and the purge valve 650. The controller may automatically actuate oxygen intake and chamber air valves to compensate for oxygen usage and fluctuations due to changes in oxygen consumption, mask leakage, and manual oxygen or chamber air addition. The controller can automatically introduce a blend or mixture of gases to control oxygen levels in the breathing loop as conditions change. For example, the controller may maintain constant oxygen levels by adjusting PPO₂ even as ambient pressure and temperature changes. The addition of pure oxygen or oxygen-concentrated gas and/or chamber air by electronic actuation can automatically adjust oxygen levels in the breathing loop without increasing rebreather internal pressure or venting breathing gas into the ambient environment. This improves safety and breathing comfort while enabling operation in chambers that cannot be vented. Furthermore, automated purging of exhalation air from the breathing loop can raise oxygen levels in the breathing loop to desired levels without relying on user lung capacity or lung strength.

The controller may be configured to provide an oxygen delivery schedule such as a desired treatment schedule or decompression schedule. In some embodiments, the oxygen treatment schedule can be based at least in part on user characteristics such as the user's height, weight, age, breathing/lung capacity, and medical needs. The oxygen treatment schedule can also account for ambient conditions and breathing loop conditions. In some implementations, the oxygen treatment schedule may provide targeted oxygen levels for a targeted duration when the user is at a certain depth. By way of an example, an oxygen treatment schedule may be a decompression schedule that requires 50% oxygen by volume at 1 ATA for 24 hours. The controller may perform the decompression schedule without having an operator manually monitor and control oxygen levels across 24 hours.

Returning to FIG. 5, the oxygen delivery and reclamation system 500 provides individualized control of oxygen level setpoints for each of the plurality of rebreather devices 520 adapted for a plurality of users. Different users may require different oxygen concentrations and treatments. Some users may be more sensitive to higher levels of oxygen during treatment or decompression. Some users may require different decompression schedules. Pure oxygen or oxygen-concentrated gas may be delivered from the oxygen-enriched gas supply source 510 to the plurality of rebreather devices 520. Delivery of the pure oxygen or oxygen-concentrated gas may be regulated by an electronically or mechanically actuated valve 516. The valve 516 regulates delivery of oxygen or oxygen-concentrated gas in an automated and controlled manner to one or more of the plurality of rebreather devices 520. The oxygen or oxygen-concentrated gas is delivered to the rebreather devices 520 through the one or more gas delivery lines 502. Oxygen levels for each user can be influenced by controlling actuation of the valve 516 for the corresponding rebreather device 520. Oxygen levels for each user can also be influenced by controlling actuation of chamber air intake valves for diluting oxygen with chamber air addition and actuation of purge valves for purging exhalation air from the breathing loop. Thus, a desired oxygen level or specified oxygen level setpoint can be achieved by controlling delivery of high-purity oxygen via the valve 516 and also controlling delivery of chamber air and controlling purging of exhalation air for each breathing loop. This ensures desired oxygen levels or specified oxygen level setpoints are met for each breathing loop. In some embodiments, the oxygen level setpoint may be between about 21% oxygen by volume and about 100% oxygen by volume, at least about 50% oxygen by volume, or at least about 90% oxygen by volume.

Each rebreather device 520 in the chamber 550 receives oxygen-concentrated gas and discharges exhalation air. The exhalation air is generally not discharged into the chamber 550 except through manual control of a relief valve to avoid over-pressurization in the breathing loop. The exhalation air may be recycled directly back to the user within the breathing loop by removing carbon dioxide using a carbon dioxide scrubber, thereby providing scrubbed exhalation air for inhalation. Alternatively, the exhalation air may be purged. As the exhalation air accumulates in the rebreather device 520 and collects in the one or more purge lines 504, one or both of the recycle pump 524 and the electronically or mechanically actuated valve 526 may purge the exhalation air. In some embodiments, the valve 526 may be a simple pressure relief diaphragm/valve depending on system design. Purging of the exhalation air can be done with or without assistance from the user's lungs. The exhalation air is purged from the breathing loop in a controlled and automated manner. The exhalation air may be purged to the recycle line 506 for oxygen reclamation. In the alternative, the exhalation air may be purged to atmosphere or to an environment outside the chamber 550. The recycle pump 524 in combination with the valve 526 may assist in pulling a vacuum on the one or more purge lines 504 for purging the exhalation air from the rebreather devices 520. A vacuum or pressure differential generated in the recycle line 506 pulls the exhalation air from the one or more purge lines 504.

In some implementations, the exhalation air is optionally treated by a hygiene/trace contaminant removal system 530. The hygiene/trace contaminant removal system 530 may serve to clean the exhalation air of contaminants. Prior to oxygen reclamation, the exhalation air may be treated to remove organics, acids, and other contaminant gases. The hygiene/trace contaminant removal system 530 may be downstream of the rebreather devices 520 and upstream of the recycle pump 524.

When purging the exhalation air for recycling, the exhalation air may be optionally collected in a buffer/accumulator tank 560. The buffer/accumulator tank 560 may serve to smooth out a feed of the exhalation air to a carbon dioxide scrubber 570 and/or the oxygen concentrator 580. An electronically or mechanically actuated valve 536 may provide controlled delivery of the exhalation air from the buffer/accumulator tank 560 to the carbon dioxide scrubber 570 or oxygen concentrator 580.

In some implementations, chamber air may be fed into the recycle line 506 from the chamber 550. Pulling chamber air into the recycle line 506 may serve to regulate ambient pressure in the chamber 550. In some implementations, a controller may receive an indication that an ambient pressure of the chamber 550 is above a threshold value or an indication that an oxygen concentration/partial pressure in the chamber 550 is above a threshold value, and the controller may activate a valve to pull the chamber air from the chamber 550. One or both of a recycle pump 534 and an electronically or mechanically actuated valve 536 may pull the chamber air into the recycle line 506. The chamber air may be pulled into the recycle line 506 by a vacuum or pressure differential. The electronically or mechanically actuated valve 536 may provide controlled delivery of the chamber air from the chamber 550 to the carbon dioxide scrubber 570 or oxygen concentrator 580.

In some implementations, chamber air may be supplied separately or in combination with the exhalation air to the carbon dioxide scrubber 570 or oxygen concentrator 580. In some implementations, the exhalation air may be supplied separately or in combination with the chamber air to the carbon dioxide scrubber 570 or oxygen concentrator 580.

The oxygen delivery and reclamation system 500 may optionally include a carbon dioxide scrubber 570 upstream of the oxygen concentrator 580. The carbon dioxide scrubber 570 is configured to remove carbon dioxide from one or both of the exhalation air and the chamber air in the recycle line 506.

The oxygen concentrator 580 receives one or both of the exhalation air and the chamber air, which may or may not be scrubbed. The oxygen concentrator 580 receives the feed of exhalation air and/or chamber air from the recycle line 506. The oxygen concentrator 580 converts the exhalation air and/or chamber air into oxygen-enriched gas. In some embodiments, the oxygen concentrator takes the exhalation air and/or chamber air and outputs oxygen-enriched gas and oxygen-depleted gas. The oxygen-enriched gas may be at least about 70% oxygen by volume, at least about 80% oxygen by volume, or at least about 90% oxygen by volume. The oxygen-enriched gas may be balanced by nitrogen. The oxygen-depleted gas may contain less than about 25% oxygen by volume, less than about 22% oxygen by volume, or between about 17% and about 22% oxygen by volume. The oxygen-depleted gas may be balanced by nitrogen. The oxygen-enriched gas may be delivered to the oxygen-enriched gas supply source 510. Therefore, the oxygen concentrator 580 replenishes oxygen in purged exhalation air and/or chamber air so that high-purity oxygen can be recycled to the plurality of users. The oxygen-depleted gas may be discharged or exhausted. In some embodiments, the oxygen-depleted gas can be discharged to the chamber 550. An electronically or mechanically actuated valve 576 can control release of the oxygen-depleted gas to the chamber 550. In some embodiments, the oxygen-depleted gas can be discharged to atmosphere or to an environment outside the chamber 550. An electronically or mechanically actuated valve 586 can control release of the oxygen-depleted gas to atmosphere or to the environment outside the chamber 550. In some embodiments, the oxygen-depleted gas can be discharged to a storage source (not shown).

In some implementations, the oxygen concentrator 580 may be an electrochemical oxygen concentrator, a vacuum swing adsorption oxygen concentrator, or a pressure swing adsorption oxygen concentrator. The oxygen concentrator 580 may be compact and efficient. In some implementations, the oxygen concentrator 580 may be positioned outside the chamber 550 to preserve space inside the chamber 550. The oxygen concentrator 580 may concentrate air close to 100% oxygen by volume. For instance, the oxygen concentrator 580 may provide high-purity oxygen at a concentration of at least about 90% oxygen by volume.

In some implementations, the oxygen concentrator may be an electrochemical oxygen concentrator. An electrochemical oxygen concentrator may have no moving parts, operate silently, and be capable of self-pressurization. The electrochemical oxygen concentrator may receive a humidified air feed. The electrochemical oxygen concentrator may transport oxygen from a low concentration chamber to a high concentration chamber. The amount of oxygen transported to the high concentration chamber is proportional to the DC current applied to the electrochemical oxygen concentrator. Any portion of the air feed that is not transported to the high concentration chamber exits the electrochemical oxygen concentrator through a vent port, where a vent feed exiting through the vent port may include nitrogen, carbon dioxide, and any oxygen not captured by the electrochemical oxygen concentrator. In some embodiments, the electrochemical oxygen concentrator includes a membrane electrode assembly comprised of a perfluorosulfonic acid membrane coated with catalysts. The catalysts facilitate the following reactions and the perfluorosulfonic acid membrane conducts protons from an oxygen outlet to an air inlet.

(air inlet) ½O₂+2H⁺+2e⁻- ->H₂O

(oxygen outlet) H₂O- ->2H⁺+½O₂+2e⁻

In some other implementations, the oxygen concentrator 580 may be a molecular sieve oxygen concentrator such as a vacuum swing adsorption oxygen concentrator or pressure swing adsorption oxygen concentrator. Generally, molecular sieve oxygen concentrators use tanks filled with a material that selectively adsorbs nitrogen to separate nitrogen from an air feed. In some embodiments, the material is a zeolite. Pressure swing adsorption oxygen concentrators transport the air feed at high pressure, vent the oxygen-enriched air, and reduce the pressure to clear the adsorbed nitrogen from the zeolite. Vacuum swing adsorption oxygen concentrators operate at lower pressures and use a vacuum pump to clear the adsorbed nitrogen from the zeolite after each cycle.

The oxygen concentrator 580 may be configured to scavenge enough oxygen from exhalation air and the chamber air in the recycle line 506 to offset or substantially offset oxygen consumed by the plurality of users and leaked into the chamber 550. The oxygen generation rate of the oxygen concentrator 580 is directly proportional to the DC current supplied to an electrochemical oxygen concentrator. By way of an example, a user may consume about 1.1 standard liters per minute (SLPM) of oxygen and a mask may leak about 2.8 SLPM of oxygen to the chamber 550. About 2.8 SLPM of oxygen would be removed from the chamber 550 to be replenished by the oxygen concentrator 580 to maintain a constant volume in the chamber 550. The oxygen concentrator 580 may be configured to generate 140 standard liters per minute (SLPM) of oxygen for 35 users of the oxygen delivery and reclamation system 500, providing about 3.9 SLPM of oxygen per user.

The oxygen-enriched gas produced by the oxygen concentrator 580 is provided to the oxygen-enriched gas supply source 510. In some implementations, the oxygen delivery and reclamation system 500 further includes an oxygen tank 540 fluidly coupled to the oxygen-enriched gas supply source 510. The oxygen tank 540 supplies pure oxygen to the oxygen-enriched gas supply source 510. A mass flow controller (MFC) 546 may regulate flow of oxygen from the oxygen tank 540 to the oxygen-enriched gas supply source 510. Thus, the oxygen-enriched gas supply source 510 may deliver pure oxygen supplied from the oxygen tank 540, oxygen-enriched gas supplied from the oxygen concentrator 580, or mixtures thereof. In some embodiments, high-purity oxygen can be delivered from the oxygen-enriched gas supply source 510, where an oxygen concentration may be at least about 70% oxygen by volume, at least about 80% oxygen by volume, or at least about 90% oxygen by volume. The high-purity oxygen is delivered to the plurality of rebreather devices 520 via the one or more gas delivery lines 502. Inhalation gas received by users in the chamber 550 includes the high-purity oxygen or a mixture of the high-purity oxygen diluted with chamber air. The inhalation gas received by the users in the chamber 550 are provided at specified oxygen level setpoints. Accordingly, the oxygen delivery and reclamation system 500 can recycle oxygen in a closed loop or semi-closed loop regardless of user lung capacity or lung strength.

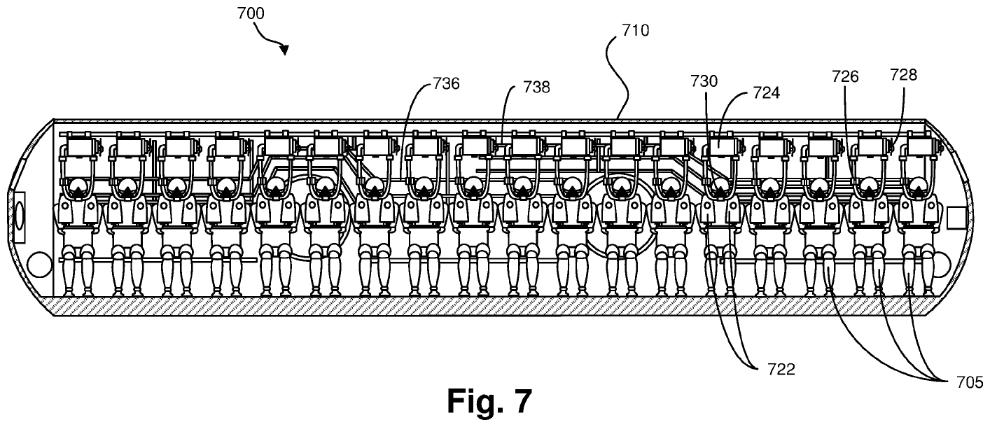

FIG. 7 shows a schematic illustration of multiple divers using example rebreather devices coupled to a multi-person oxygen delivery and reclamation system in a submarine decompression chamber according to some implementations. The submarine decompression chamber 710 is a pressurized vessel providing an enclosed volume for users 705 to undergo decompression cycles. The submarine decompression chamber 710 may hold a plurality of users 705. The submarine decompression chamber 710 further includes a plurality of rebreather devices 720 adapted for each of the plurality of users 705. Each of the rebreather devices 720 include breathing bags 722 that rest over the shoulders of the users 705. Furthermore, each of the rebreather devices 720 include a mask 730 configured to be worn by the user 705. A carbon dioxide scrubber assembly 724 of each rebreather device 720 is positioned above the users 705 and may be connected to a handrail in the submarine decompression chamber 710. Each of the rebreather devices 720 further includes a purge valve 726 connected to purge tubing 736 of the multi-person oxygen delivery and reclamation system 700. Each of the rebreather devices 720 further includes an oxygen intake valve 728 connected to oxygen tubing 738 of the multi-person oxygen delivery and reclamation system 700. Aspects of the multi-person oxygen delivery and reclamation system 700 are described in FIGS. 4 and 5, and aspects of the rebreather devices 720 are described in FIGS. 6A and 6B.

Although the foregoing disclosed systems, methods, apparatuses, processes, and compositions have been described in detail within the context of specific implementations for the purpose of promoting clarity and understanding, it will be apparent to one of ordinary skill in the art that there are many alternative ways of implementing foregoing implementations which are within the spirit and scope of this disclosure. Accordingly, the implementations described herein are to be viewed as illustrative of the disclosed inventive concepts rather than restrictively, and are not to be used as an impermissible basis for unduly limiting the scope of any claims eventually directed to the subject matter of this disclosure.

What is claimed is:

1. An oxygen delivery and reclamation system, the system comprising:
one or more rebreather devices in a chamber, each of the one or more rebreather devices comprising a mask configured to be worn by a user;
an oxygen-enriched gas supply source configured to supply an oxygen-concentrated inhalation gas upon activation to the one or more rebreather devices;
a purge line configured to purge exhalation gas from the one or more rebreather devices;
a recycle line coupled to the purge line and configured to receive the exhalation gas from the purge line; and
an oxygen concentrator coupled to the recycle line, wherein the oxygen concentrator is configured to receive the exhalation gas and output an oxygen-enriched gas to the oxygen-enriched gas supply source and an oxygen-depleted gas to an ambient environment outside the chamber, to a storage source, or to the chamber.

2. The oxygen delivery and reclamation system of claim 1, wherein the oxygen-enriched gas comprises at least 90% oxygen by volume.

3. The oxygen delivery and reclamation system of claim 1, further comprising:
a carbon dioxide (CO₂) scrubber coupled to the recycle line, wherein the carbon dioxide scrubber is configured to remove carbon dioxide from the exhalation gas prior to being received by the oxygen concentrator.

4. The oxygen delivery and reclamation system of claim 1, further comprising:
a chamber pump configured to supply chamber air from the chamber to the recycle line, wherein the oxygen concentrator is configured to receive the chamber air or a mixture of the chamber air and the exhalation gas and output the oxygen-enriched gas and the oxygen-depleted gas.

5. The oxygen delivery and reclamation system of claim 4, further comprising:
a controller configured with instructions to perform the following operations:
receive an indication that an ambient pressure or oxygen concentration/partial pressure of the chamber is above a threshold value; and
actuate an electronically or mechanically actuated valve coupled to the chamber pump to pull the chamber air into the recycle line and regulate the ambient pressure.

6. The oxygen delivery and reclamation system of claim 1, further comprising:
a purge pump configured to generate a vacuum in the recycle line and pull the exhalation gas from the purge line to the recycle line.

7. The oxygen delivery and reclamation system of claim 6, further comprising:
an atmospheric line coupled to the purge line and configured to vent the exhalation gas to the ambient environment outside the chamber or to the storage source, wherein the exhalation gas is supplied to the recycle line or the atmospheric line via an electronically or mechanically actuated valve.

8. The oxygen delivery and reclamation system of claim 1, wherein the chamber is a pressurized vessel.

9. The oxygen delivery and reclamation system of claim 1, further comprising:
an oxygen tank coupled to the oxygen-enriched gas supply source and configured to supply oxygen to the oxygen-enriched gas supply source, wherein the oxygen-concentrated inhalation gas includes the oxygen or a mixture of the oxygen and the oxygen-enriched gas.

10. The oxygen delivery and reclamation system of claim 1, wherein each of the one or more rebreather devices further comprises: an inhale breathing bag coupled to an inlet of the mask, and an exhale breathing bag coupled to an outlet of the mask.

11. The oxygen delivery and reclamation system of claim 1, further comprising:
a controller configured with instructions to perform the following operations:
receive an input for a desired oxygen concentration to be supplied to a user of one of the one or more rebreather devices; and
actuate an oxygen intake valve fluidly coupled to the oxygen-enriched gas supply source and a chamber air intake valve fluidly coupled to the chamber to deliver the desired oxygen concentration to a rebreather device associated with a user.

12. The oxygen delivery and reclamation system of claim 11, wherein the controller is further configured with instructions to perform the following operations:
actuate a purge valve coupled to the purge line, independent of user lung capacity and lung strength, to purge the exhalation gas independently from the one or more rebreather devices.

13. The oxygen delivery and reclamation system of claim 1, wherein the one or more rebreather devices comprises a plurality of rebreather devices comprising masks worn by a plurality of users.

14. The oxygen delivery and reclamation system of claim 1, wherein the oxygen concentrator is an electrochemical oxygen concentrator.

15. The oxygen delivery and reclamation system of claim 1, further comprising:
a hygiene/trace contaminant removal device coupled to the purge line and configured to remove contaminants from the exhalation gas prior to reaching the recycle line.

16. A method of delivering and reclaiming oxygen, the method comprising:
receiving an input for a desired oxygen concentration to be supplied to a user of a rebreather device inside a chamber;
delivering an inhalation gas to the user at the desired oxygen concentration from an oxygen-enriched gas supply source;
purging exhalation gas from the rebreather device by a vacuum generated in a purge line coupled to the rebreather device;
recycling the exhalation gas to an oxygen concentrator; and
producing, by the oxygen concentrator, an oxygen-enriched gas that is supplied to the oxygen-enriched gas supply source.

17. The method of claim 16, further comprising:
producing, by the oxygen concentrator, an oxygen-depleted gas that is delivered to an ambient environment outside the chamber, to a storage source, or to the chamber.

18. The method of claim 16, further comprising:
pulling chamber air from the chamber into a recycle line fluidly coupled to the oxygen concentrator; and
supplying the chamber air to the oxygen concentrator, wherein the oxygen-enriched gas is produced by one or both of the chamber air and the exhalation gas.

19. The method of claim 16, wherein the oxygen-enriched gas comprises at least 90% oxygen by volume.

20. The method of claim 16, further comprising:
venting the exhalation gas from the rebreather device to an ambient environment outside the chamber or to a storage source.

21. A method of monitoring and controlling gas composition delivery, the method comprising:
receiving, at a computer device, a desired target for a concentration/partial pressure of a first gas to be delivered to a user of a gas control system;
receiving, at the computing device, data regarding a concentration/partial pressure of the first gas in the gas control system, a concentration/partial pressure of a second gas in the gas control system, and one or more internal conditions associated with a user of the gas control system over a period of time;
correlating, at the computer device, changes in the concentration/partial pressure of the second gas and/or changes in the one or more conditions associated with the user with changes in the concentration/partial pressure of the first gas; and
calculating, using the computing device, an amount of the first gas or a third gas to be delivered or regulated to the user in response to changes in the concentration/partial pressure of the second gas and/or changes in the one or more conditions associated with the user in order to reach the desired target for the concentration/partial pressure of the first gas to be delivered to the user.

22. The method of claim 21, wherein the first gas is oxygen and the second gas is carbon dioxide.

23. The method of claim 22, further comprising:
regulating the amount of the first gas to be delivered to the user using one or more electronically or mechanically actuated valves by oxygen addition, chamber air addition, and/or exhalation air purging.

24. The method of claim 23, wherein regulating the amount of the first gas comprises controlling the amount of the first gas delivered to the user according to a treatment schedule.

25. The method of claim 21, wherein the one or more conditions associated with the user comprises at least one of: internal pressure, ambient pressure, volume, temperature, and relative humidity.

26. The method of claim 21, wherein the desired target for the concentration/partial pressure of the first gas is reached in response to changes in ambient pressure and/or internal pressure.

27. The method of claim 21, wherein receiving the data comprises receiving the data from a sensor assembly comprising a plurality of sensors configured to reliably operate even at pressures up to about 3 atmospheres absolute.
